(19) United States
(12) Patent Application Publication
(10) Pub. No.: US 2024/0287442 A1
(43) Pub. Date: Aug. 29, 2024

(54) MICROALGAE STRAIN CHLORELLA SP. VADA 2020

(71) Applicant: CHLO & RELLA CORP, Sarasota, FL (US)

(72) Inventors: Denis Aleksandrovich POSTOVOY, Novosibirsk (RU); Viktor Vladimirovich OSKOLKOV, Novosibirsk (RU); Alexandr Vasilevich PILIGAEV, Novolokti (RU)

(21) Appl. No.: 18/568,297

(22) PCT Filed: May 19, 2022
(86) PCT No.: PCT/RU2022/050159

§ 371 Date: Dec. 8, 2023

(30) Foreign Application Priority Data
Jun. 30, 2021 (RU) 2021119256

(51) Int. Cl. C12N 1/12; C12R 1/89

(52) U.S. Cl. CPC C12N 1/125; C12R 2001/89

(57) ABSTRACT

The invention relates to the field of biotechnology. A strain of microalgae Chlorella sp. VADA 2020 was isolated from water samples of Lake Tarmakul of the Chanovsky district of the Novosibirsk region and deposited in the Collection of Microalgae of the Plant Physiology Institute, RAS, under the registration number IPPAS C-2048. Available nutrient media are used for cultivating the strain. Namely, biomass with a protein content of 48.1 wt. %, and with a content of unsaturated and monounsaturated fatty acids in an amount of 59.1 wt. % (of the total content), is produced that can be used as raw materials to produce food additives. The invention makes it possible to produce biomass with a high content of protein and unsaturated fats, suitable for use for food purposes. 2 tbl, 2 ex.

The invention relates to microbiology and biotechnology and is a new strain of unicellular green microalgae Chlorella sp. VADA 2020, which can be used to produce food-grade biomass.

Microalgae are recognized as a promising source of biomass with a high content of valuable chemicals suitable for use in the food industry. Microalgae biomass contains a significant amount of lipids (with polyunsaturated ω-3 and ω-6 acids), proteins, chlorophyll, carotenoids, vitamins, minerals, and probiotic compounds that improve health (Hayes M. et al., Microalgal proteins for feed, food and health. II Microalgae-based biofuels and bioproducts: from feedstock cultivation to end-product II 2017. p. 347-368). However, not all strains have an optimal chemical composition and are suitable to produce biomass with high yield under laboratory and industrial cultivation conditions. Another problem of cultivation is the production of pure biomass and the possibility of its long-term storage in suspension without contamination by foreign microflora.

There is a strain of unicellular green microalgae Chlorella vulgaris IFR C-111, used for producing biomass and corresponding to the requirements of industrial cultivation. The cell wall consists of inner and outer layers, and the outer layer contains extensive myofibrillar material (patent RU 1751981, publ. 10.02.1997).

The disadvantages of the strain Chlorella vulgaris IFR C-111 are the asynchronous cell life cycle, seasonality (from May to December), sensitivity to the composition of the nutrient medium, a strong and thick cell wall, and a narrow range of cultivation temperatures (26-36° C.).

There is a strain 132-1 of the unicellular green microalgae Chlorella vulgaris f. Suboblonga has the following characteristics: cells are ellipsoid, length 2.8-6.1 μm and width 1.7-5.5 μm, with the formation of autospores 8.0 and 5.5 μm, respectively. The chloroplast is wide, annular, open, or trough-shaped. The position of the chloroplast can be oriented longitudinally or transversely with respect to the long axis of the cell. Autospores are arranged in 2-4, less often in 8, irregularly ellipsoidal immediately after release. The strain 132-1 is stored at the Botanical Institute of the Russian Academy of Sciences and is a museum culture (Andreeva V. M. The genus Chlorella. Morphology, systematics, principles of classification. L.: “Nauka”, Leningrad. otd., 1975.-110 p.).

There is a strain of unicellular green microalgae Chlorella vulgaris N A-29, intended for producing biomass, when cultivated on Tamiya medium at 42° C., a dry biomass yield of 6.38 g/I was achieved. The microalgal strain Chlorella vulgaris N A-29 was obtained by mutation-selection using nitrosomethylurea from wild strain 132. (Patent RU 2005777 C1, publ. 14.06.1991).

The disadvantage of the strain Chlorella vulgaris N A-29 is a narrow temperature optimum, adaptability to conditions only for a hot climate, such as Central Asia, and cultivation in special industrial-type apparatuses (glass-pipe installations).

Closest to the claimed prototype strain is a strain of microalgae Chlorella vulgaris VKPM A1-24, which was cultivated on a nutrient medium with the inclusion of grain material (hulled oats) in its composition to obtain a carbon dioxide solution. As a result of selection, a strain was selected that has a thin cell wall, capable of intensively reproducing biomass in a synchronous cultivation mode and maintaining monoculture during production (Patent RU 2644653 C1, publ. Feb. 13, 2018).

The disadvantage of the strain Chlorella vulgaris VKPM A1-24 is the high risk of contamination of bacterial microflora when cultivated using a carbon dioxide solution prepared based on naked oats, which limits the possibility of using the strain for food purposes.

The aim of the invention is to obtain a new strain of microalgae Chlorella sp., which has a high growth rate under aseptic conditions and the optimal chemical composition of the biomass suitable for food use in the form of a suspension or dry powder.

The technical result: biomass production with a high content of protein and unsaturated fats suitable for food purposes.

This aim is achieved by the proposed strain of microalgae Chlorella sp. VADA 2020, which has a high specific growth rate of biomass with a high content of protein and unsaturated and monounsaturated fatty acids, suitable for food use.

The strain of microalgae Chlorella sp. VADA2020 was isolated from water samples from Lake Tarmakul in the Chanovsky district of the Novosibirsk region and deposited in the Microalgae Collection of the IFR RAS under the registration number IPPAS C-2048.

The strain of microalgae Chlorella sp. VADA 2020 has the following morphological and physiological characteristics.

Cells are round, spherical, with a thin cell wall without mucus, 3-10 μm in size, with a single pyrenoid, 1 μm in size, reproduction occurs by 2-4 autospores. On agar BBM media forms green colonies with a smooth edge. The strain is photoauto- and mixotrophic. The cultivation temperature is from 15 to 35° C. The strain grows on a mineral BBM medium and does not require additional carbon dioxide supply to the medium for growth. A sufficient period of illumination for growth is up to 16-18 hours per day. The strain is not prone to sedimentation, both in culture and on the walls of the cultivation unit, and remains axenic during storage. The strain grows regardless of the season of the year.

The resulting strain, in comparison with the previously described strains, has an increased content of protein (48.1%) and lipids with monounsaturated fatty acids and polyunsaturated fatty acids. The total content of these fatty acids, determined by gas chromatography-mass spectrometry, is 59.1%, as shown in Table 2, which makes it possible to produce biomass with a high content of unsaturated fats, which is advantageous forfood use.

The invention is illustrated by the following examples:

### EXAMPLE 1

A flat panel glass photobioreactor 800 mm×312 mm×500 mm (length×width×height) with an effective volume of 100 L was used for batch cultivation under indoor conditions. An air mixture with 1.5% CO2 was supplied with a rotameter at a constant flow rate of 200 L h-1 through a porous tube in the bottom of the chamber. Inoculation was carried out with a culture in the exponential growth phase, with an initial concentration of microalgal cells of 3 million cells/ml. Chlorella sp. VADA 2020 was cultured in BBM medium for 5 days at a temperature of 30±1° C. The photobioreactor was illuminated from both sides with LED panels at 9850 kLx. To estimate the growth characteristics and composition of the biomass during cultivation, aliquots of 500 ml of the cell suspension were taken, then the cells were washed and precipitated by centrifugation at 10,000 g for 10 min. The resulting biomass was dried to constant weight and used for analysis of the chemical composition.

Table 1 presents data on growth characteristics and biomass composition of the microalgal strain Chlorella sp. VADA 2020 with growth in BBM media.

TABLE 1

| | | | | | Total |
|---|---|---|---|---|---|
| | | Biomass | Total protein | Total lipid | carbohydrate |
| | | productivity, | content, % | content, % | content, % |
| Strain | μ (d−1) | mg L−1 d−1 | (dry weight) | (dry weight) | (dry weight) |
| IPPAS C- | 0.7 ± 0.1 | 245.4 ± 8.6 | 48.1 ± 1.6 | 22.6 ± 0.8 | 16.5 ± 1.8 |
| 2048 | | | | | |

Table 1 shows that the strain is characterized by a high biomass productivity (245.4±8.6 mg I⁻¹ day⁻¹) and with a high protein content (48.1±1.6%), as well as a high specific growth rate of 0.7±0.1 day⁻¹. The proposed strain produces biomass with protein content comparable to that produced by the C. vulgaris strain (47.82% protein) disclosed in a study (Tokusoglu, O. et al., Biomass nutrient profiles of three microalgae: Spirulina platensis, Chlorella vulgaris, and Isochrisis galbana II J. of Food Science.-2003.-V. 68.-N. 4.-P. 1144-1148) and a strain of C. vulgaris with a protein content of 46%, described in the work (Seyfabadi, J. et al. Protein, fatty acid, and pigment content of Chlorella vulgaris under different light regimes II Journal of Applied Phycology.-2011.-V. 23.-N. 4.-P. 721-726).

### EXAMPLE 2

The fatty acid methyl esters composition was analyzed by gas chromatography mass-spectrometry method using Agilent 7000B GC/MS with a ZB-WAX column (30 m×0.25 mm) with a temperature gradient from 100° C. to 260° C. at an increase rate of 12° C. min⁻¹. The injector temperature was set at 260° C. Carrier gas (helium) flow was set to 1.2 cm3 min⁻¹. Peaks of fatty acids were identified by using the NIST'11 library, and the relative amount of individual fatty acid was calculated by the integrated area percentage from the total amount of fatty acids. Experiments were carried out in triplicate, and the data are expressed as mean±SD.

Table 2 shows the fatty acid composition of microalgae Chlorella sp. VADA 2020 in photoautotrophic cultivation according to gas chromatography-mass spectrometry analysis.

| Fatty acid | % from total amount |
|---|---|
| C14:0 | 1.2 ± 0.1 |
| C16:0 | 20.1 ± 1.4 |
| C16:1 | 1.5 ± 0.2 |
| C16:2 | 6.2 ± 0.6 |
| C16:3 | 7.1 ± 0.3 |
| C18:0 | 18.9 ± 0.8 |
| C18:1 | 17.8 ± 1.0 |
| C18:2 | 15.3 ± 0.9 |
| C18:3 | 10.7 ± 2.1 |
| C20:0 | 0.4 ± 0.1 |
| C20:1 | 0.5 ± 0.1 |
| ΣSFA | 40.6 |
| ΣPUFA | 39.3 |
| ΣMUFA | 19.8 |
| ΣPUFA + MUFA | 59.1 |

In table 2, the following abbreviations are used: the first value after the symbol “C” is the number of carbon atoms, the second value after the colon is the number of double bonds in the fatty acid molecule; SFA-saturated fatty acids, PUFA-polyunsaturated fatty acids, MUFA-monounsaturated fatty acids.

Table 2 shows that 11 fatty acids with a carbon chain length from C14 to C20 were found in the lipid composition of the studied strain. The main unsaturated fatty acids of the biomass of Chlorella sp. VADA 2020 were C16:2, C16:3, C18:1, C18:2 and C18:3 acids. The total content of all PUFAs and MUFAs in the studied strain after 5 days of cultivation was 59.1%. This value is comparable to previously published fatty acid values for Chlorella microalgae promising for food use (Matos, et al., Chemical Characterization of Six Microalgae with Potential Utility for Food Application. J. Am. Oil Chem. Soc, 93: 963-972). Thus, the proposed strain of Chlorella sp. VADA 2020 can be used to produce biomass with a high content of unsaturated fats suitable for food purposes.

What is claimed is:

1. The strain of microalgae Chlorella sp. VADA 2020, deposited in the IFR RAS Microalgae Collection under registration number IPPAS C-2048, producing food-grade biomass.
